(19) United States
(12) Patent Application Publication
(10) Pub. No.: US 2017/0325844 A1
(43) Pub. Date: Nov. 16, 2017

(54) UTERINE MANIPULATOR

(71) Applicant: Covidien LP, Mansfield, MA (US)

(72) Inventor: Scott J. Prior, Shelton, CT (US)

(21) Appl. No.: 15/466,201

(22) Filed: Mar. 22, 2017

Related U.S. Application Data
(60) Provisional application No. 62/334,074, filed on May 10, 2016.

(51) Int. Cl. A61B 17/42; A61B 1/06; A61B 1/303; A61B 17/00

(52) U.S. Cl. CPC A61B 17/4241; A61B 17/00234; A61B 1/0676; A61B 1/303

(57) ABSTRACT

A uterine manipulator is described that includes an elongated, hollow shaft having a proximal end and a distal end. The hollow shaft defines a channel and has a non-opaque distal tip. A handle is positioned on a proximal portion of the shaft and an inflatable balloon is supported on a distal portion of the shaft. The inflatable balloon is transitional from a deflated state to an inflated state. A cervical cup is movably positioned along the shaft from a distal position to a proximal position. The channel of the shaft is dimensioned to receive an endoscope to facilitate visualization of a body lumen through the non-opaque distal tip of the shaft. In embodiments, the cervical cup defines a rim that is positioned to abut the inflatable balloon when the cervical cup is in its distal position. The rim includes a plurality of distally facing lights that function to identify placement of the cervical cup in relation to the cervix.

### CROSS-REFERENCE TO RELATED APPLICATIONS

This application claims the benefit of and priority to U.S. Provisional Patent Application No. 62/334,074 filed May 10, 2016, the entire disclosure of which is incorporated by reference herein.

### BACKGROUND

### 1. Technical Description

The present disclosure relates to a uterine manipulator and its method of use. More particularly, the present disclosure relates to a uterine manipulator configured to provide improved visualization during passage of the uterine manipulator through the vagina and cervix to facilitate proper placement of a distal portion of the uterine manipulator within the uterus, and its method of use.

### 2. Background of Related Art

Uterine manipulators are conventionally used during laparoscopic hysterectomies to mobilize and position the vagina and the cervix to facilitate their separation and to allow for removal of the uterus after colpotomy is performed. Typically, uterine manipulators include a handle, a shaft extending distally from the handle, an inflatable balloon supported on the end of the shaft opposite the handle, and a cervical cup supported on the shaft proximally of the inflatable balloon. In use, the inflatable balloon is advanced through the vagina and cervix and is positioned within the uterus in a deflated state. Once positioned within the uterus, the inflatable balloon is inflated to secure the uterine manipulator within the uterus. The uterine manipulator, as its name suggests, is subsequently used to manipulate the uterus during separation and removal of the uterus from the vagina.

During placement of known uterine manipulators into the uterus, visualization of the vagina, cervix and uterus may be obscured thus rendering proper placement of the cervical cup and inflatable balloon more difficult. A continuing need exists in the art for a uterine manipulator that facilitates improved visualization of the vaginal canal and cervix during placement of the uterine manipulator within the uterus.

### SUMMARY

One aspect of the present disclosure is directed to a uterine manipulator including an elongated, hollow shaft having a proximal end and a distal end. The hollow shaft defines a channel and has a non-opaque distal tip. A handle is positioned on a proximal portion of the shaft and an inflatable balloon that is transitional from a deflated state to an inflated state is supported on a distal portion of the shaft. A cervical cup is also movably positioned along the shaft. The channel of the shaft is dimensioned to receive an endoscope to facilitate visualization of a body lumen through the non-opaque distal tip of the shaft.

In embodiments, the cervical cup is movably positioned along the shaft from a distal position in close approximation with the inflatable balloon to a proximal position spaced from the inflatable balloon.

In some embodiments, the cervical cup includes a rim that is positioned to engage the inflatable balloon when the balloon is in the inflated state and the cervical cup is in the distal position.

In certain embodiments, the rim supports a plurality of lights.

In embodiments, the plurality of lights is positioned in an annular array about the rim.

In embodiments, the inflatable balloon is lubricated.

In embodiments, the uterine manipulator further includes a locking member positioned on the handle that is operable to lock the cervical cup in relation to the shaft.

Another aspect of the present disclosure is directed to a method of performing a surgical procedure that includes inserting a distal portion of a uterine manipulator into a patient's vaginal opening, the uterine manipulator including a hollow shaft defining a channel having a distal portion supporting an inflatable balloon and a non-opaque distal tip; visualizing a vaginal canal during insertion through the non-opaque distal tip with an endoscope positioned within the hollow shaft; advancing the inflatable balloon past the patient's cervix into the uterus; and inflating the balloon within the uterus to secure the uterus to the distal portion of the uterine manipulator.

In some embodiments, the method further includes positioning the inflatable balloon in close approximation with the cervical cup prior to inserting the distal portion of the uterine manipulator into the patient's vaginal opening.

In certain embodiments, the method further includes positioning the cervical cup in abutting relation to a cervix of the patient.

In embodiments, the method further includes illuminating lights positioned on a distal rim of the cervical cup.

In some embodiments, the method further includes inserting a second endoscope through the patient's abdomen to a location to visualize the lights of the rim of the cervical cup at the posterior vaginal fornix to confirm that the rim is properly placed in relation to the cervix.

In certain embodiments, the method further includes inserting a cutting device through the second endoscope and cutting tissue the directly over the lights to separate the patient's uterus from the cervix.

Another aspect of the present disclosure is directed to a uterine manipulator including an elongated shaft having a proximal end and a distal end and a handle assembly positioned on a proximal portion of the shaft. A cervical cup is movably positioned along the shaft from a distal position to a proximal position. The cervical cup includes a body having a rim supporting a plurality of lights.

In embodiments, the plurality of lights is positioned in an annular array about the rim.

In some embodiments, an inflatable balloon is supported on the shaft distally of the cervical cup. The inflatable balloon being transitional from a deflated state to an inflated state.

In certain embodiments, the elongated shaft defines a channel and has a non-opaque distal tip. The channel is dimensioned to receive an endoscope to facilitate visualization of a body lumen through the non-opaque distal tip of the elongated shaft.

In embodiments, the cervical cup defines a cavity and a tubular extension positioned about the shaft and extending towards the handle assembly. The rim of the cervical cup may be positioned to engage the inflatable balloon when the balloon is in the inflated state and the cervical cup is in the distal position.

In some embodiments, a first hand grip is supported on a proximal portion of the elongated shaft and a second hand grip is supported on a proximal portion of the tubular extension. The first hand grip is movable in relation to the second hand grip to move the body of the cervical cup axially in relation to the inflatable balloon.

### BRIEF DESCRIPTION OF THE DRAWINGS

Various embodiments of the presently disclosed uterine manipulator are described herein below with reference to the drawings, wherein:

FIG. 1 is a side perspective view of an exemplary embodiment of the presently disclosed uterine manipulator with an inflatable balloon in a deflated state and a cervical cup in its distal-most position on a shaft of the uterine manipulator;

FIG. 1A is a side perspective view of a distal portion of the uterine manipulator shown in FIG. 1 with the inflatable balloon in the deflated state;

FIG. 2 is side perspective, cross-sectional view taken along section line 2-2 of FIG. 1A with the inflatable balloon in an inflated state;

FIG. 3 is a side, partial cross-sectional view of the distal portion of the uterine manipulator shown in FIG. 2 as the uterine manipulator is inserted through the vaginal opening into the vagina with the inflatable balloon in an inflated state;

FIG. 4 is a side, partial cross-sectional view of the distal portion of the uterine manipulator shown in FIG. 1 with the inflatable balloon positioned within the uterus in the deflated state and the cervical cup positioned to abut the cervix;

FIG. 5 is a side, partial cross-sectional view of the distal portion of the uterine manipulator shown in FIG. 2 with the inflatable balloon positioned within the uterus in the inflated state and the cervical cup positioned to abut the cervix;

FIG. 6 is a side, partial cross-sectional view of the distal portion of the uterine manipulator shown in FIG. 2 with the inflatable balloon positioned within the uterus in an inflated state, the cervical cup positioned to abut the cervix, and an endoscope positioned through the abdomen to a position adjacent the cervix to facilitate visualization of the location of the cervical cup at the posterior fornix; and

FIG. 7 is a side, partial cross-sectional view of the distal portion of the uterine manipulator shown in FIG. 2 with the inflatable balloon positioned within the uterus in an inflated state, the cervical cup positioned to abut the cervix, and the abdominal endoscope positioned adjacent the cervix to facilitate visualization of the location of the cervical cup at the posterior fornix as the cervix is separated from the uterus using a surgical tool positioned through the abdominal endoscope.

### DETAILED DESCRIPTION OF EMBODIMENTS

The presently disclosed uterine manipulator will now be described in detail with reference to the drawings in which like reference numerals designate identical or corresponding elements in each of the several views. In this description, the term “proximal” is used generally to refer to the portion of the apparatus that is closer to a clinician, while the term “distal” is used generally to refer to the portion of the apparatus that is farther from the clinician. In addition, the term “endoscopic” is used generally to refer to endoscopic, laparoscopic, arthroscopic, and any other surgical procedure performed through a small incision or a cannula inserted into a patient's body. Finally, the term clinician is used generally to refer to medical personnel including doctors, nurses, and support personnel.

The presently disclosed uterine manipulator is provided to improve visualization within lumens of the body as the uterine manipulator is inserted through the vagina and cervix and into the uterus, and during separation of the uterus from the cervix. In embodiments, the presently disclosed uterine manipulator includes a flexible, hollow shaft, a handle assembly positioned on a proximal portion of the flexible shaft, an inflatable balloon positioned on a distal portion end of the flexible shaft, and a cervical cup that is movable along the flexible shaft between the inflatable balloon and the handle. The distal end of the flexible shaft is covered by a clear, non-opaque tip. The flexible shaft is dimensioned and configured to receive an endoscope or hysteroscope that allows for visualization of the vagina and the cervix through the tip as the uterine manipulator is advanced towards the uterus. The cervical cup includes an annular rim that is positioned about the shaft and is configured to abut the cervix. The annular rim supports a plurality of lights, e.g., light emitting diodes (LEDs). The lights are visible through the cervix from a position externally of the cervix to facilitate proper placement of the cervical cup in relation to the cervix prior to separation of the cervix from the uterus and to identify to a clinician the cutting location.

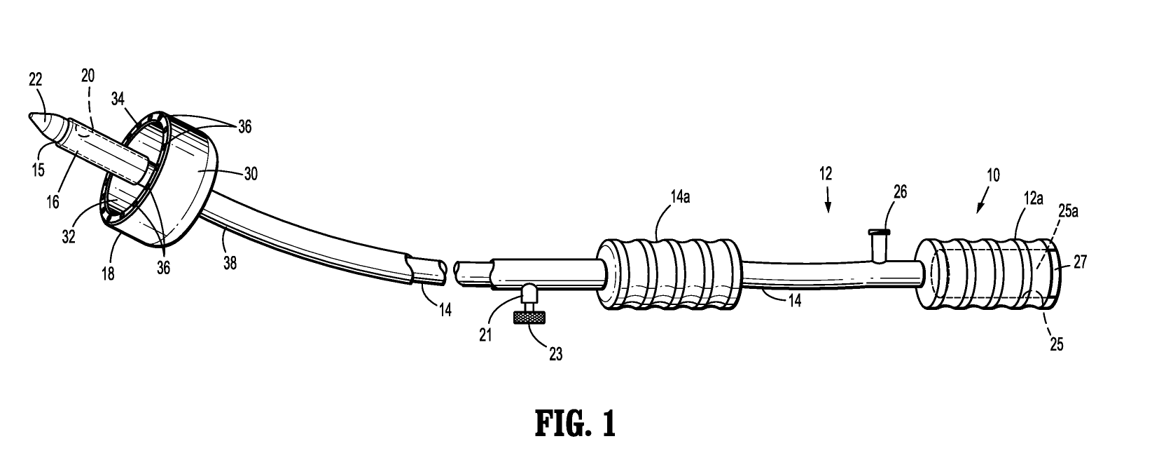

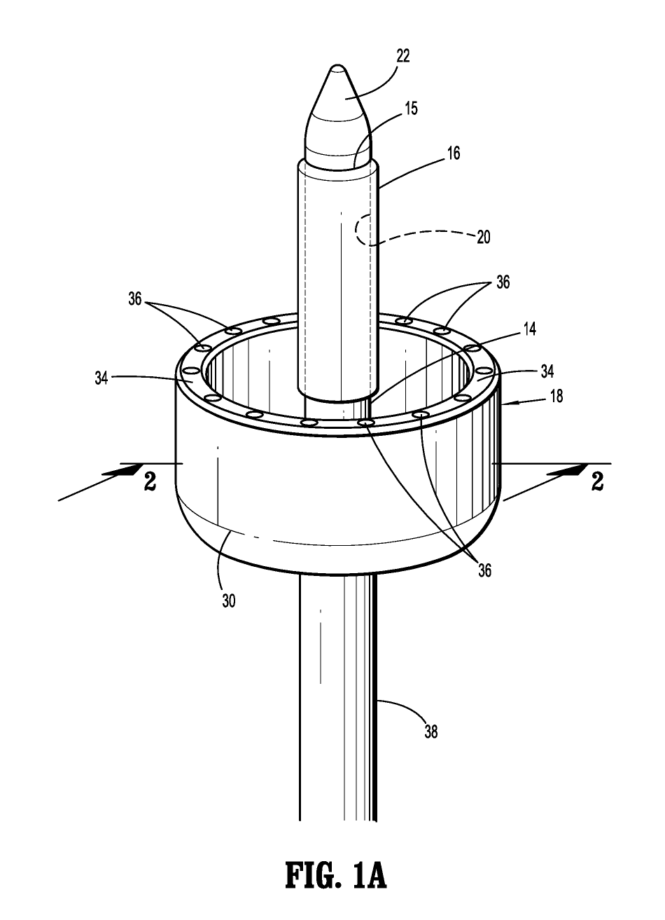

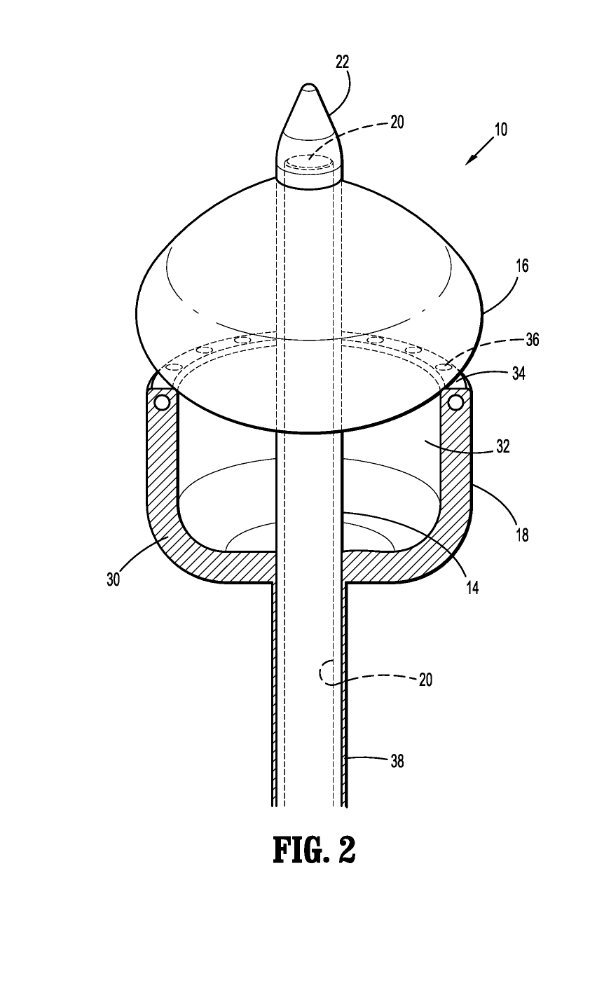

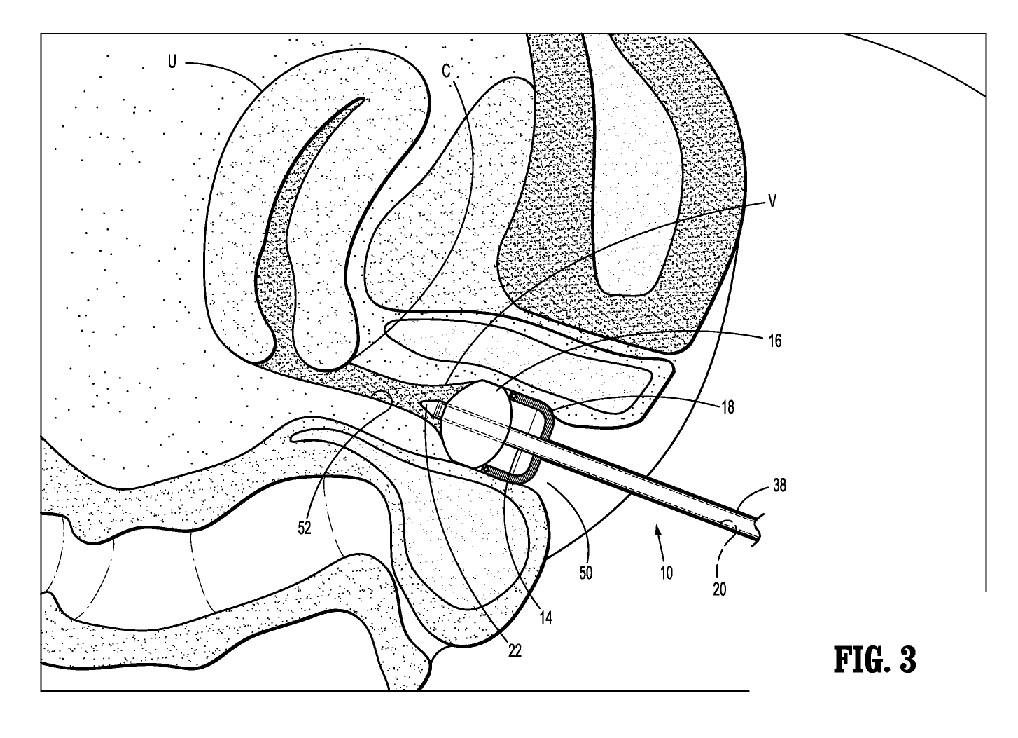

Referring to FIGS. 1-2, the presently disclosed uterine manipulator shown generally as 10 includes a handle assembly 12 (FIG. 1A), an elongated, flexible shaft 14, an inflatable balloon 16, and a cervical cup 18. The flexible shaft 14 is hollow and receives an endoscope or hysteroscope 20, e.g., a 5 mm endoscope. A distal end of the flexible shaft 14 defines an opening 15 that receives a clear or non-opaque tip 22. Alternatively, the clear tip 20 may be formed integrally with the flexible shaft 14. In embodiments, the tip 20 may have a tapered configuration to facilitate passage through the vagina “V” and cervix “C” and into the uterus. “U” (FIG. 3). The tip 20 should have a smooth, atraumatic configuration to minimize potential injury to internal organs.

The inflatable balloon 16 is supported on a distal portion of the shaft 14 adjacent the tip 22. The balloon 16 is inflatable from a deflated state shown in FIG. 1A to an inflated state shown in FIG. 2 by injecting a fluid, such as saline, water or air, into a port 26 positioned on a proximal portion of the uterine manipulator 10. Injection of fluid into the port 26 may be provided by a syringe or any other known injection device. In embodiments, the balloon 16 may be formed of a variety of different materials known in the art including polytetrafluroethylene (PTFE). In some embodiments, the balloon 16 may be impregnated or coated with a lubricious material as is known in the art to facilitate atraumatic insertion of the distal portion of the uterine manipulator 10 through a body lumen, e.g., the vaginal canal. See, e.g., U.S. Pat. No. 7,951,413 which is incorporated herein in its entirety by reference.

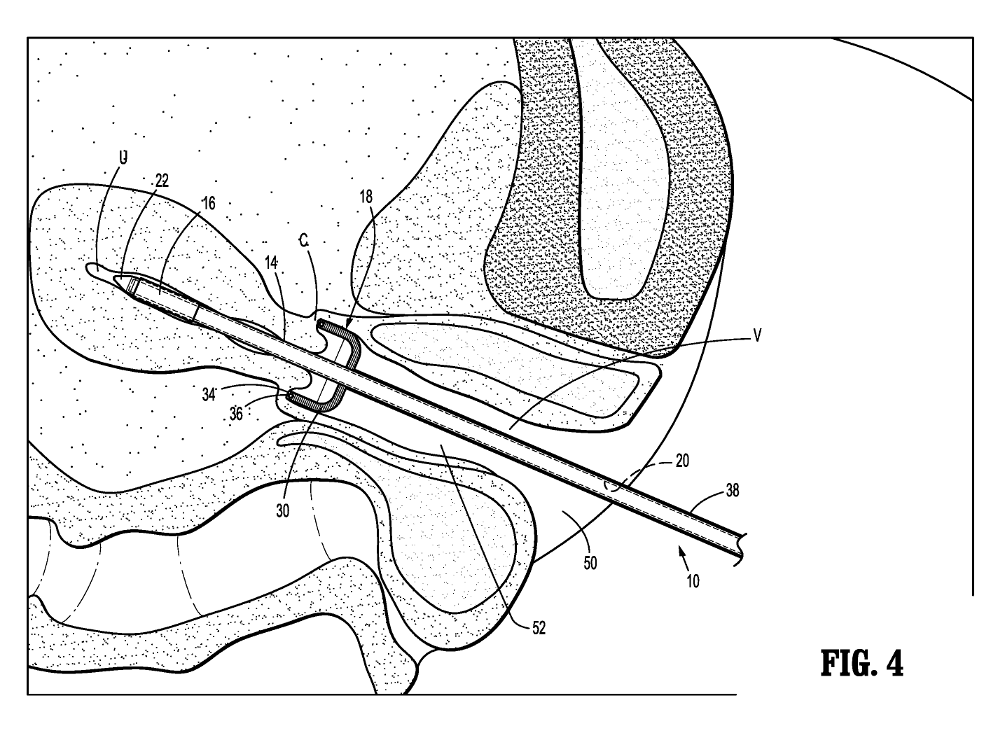

The cervical cup 18 is positioned about the outer surface of the shaft 14 and includes a body 30 defining a cavity 32 and having an annular, distally facing rim 34. The body has a tubular extension 38 that extends proximally towards the handle 12. As discussed in further detail below, the tubular extension 38 slides about the shaft 14 from a distal-most position (FIG. 3) to a proximal-most position (FIG. 4) to facilitate repositioning of the cervical cup 18 in relation to the inflatable balloon 16. In its distal-most position (FIG. 3), the rim 34 is positioned in close approximation with the proximal surface of the balloon 16 when the balloon 16 is in the inflated state (FIG. 2). In its proximal-most position along the shaft 14, the cervical cup 18 is spaced a distance from the balloon 16 sufficient to allow the balloon to be positioned within the uterus while the rim 34 of the cervical cup 18 is in abutting relationship with the cervix “C”. Although the body 30 is shown to be substantially cylindrical, it is envisioned that the body 30 may assume other configurations including frustoconical, oval, etc. . . . .

The handle assembly 12 includes a hand grip 12a that is secured to the proximal portion of the shaft 14 and a hand grip 14a that is secured to the proximal portion of the tubular extension 38 of the cervical cup 18. The hand grips 12a and 14a are movable in relation to each other to reposition the cervical cup 18 in relation to the inflatable balloon 16. A locking device 21 is supported on the tubular extension 38 and is operable to secure the position of the body 30 of the cervical cup 18 in relation to the balloon 16.

In embodiments, the locking device 21 includes a set screw 23 that is threaded to the tubular extension 38. The set screw 23 is rotatable into contact with the shaft 14 to secure the shaft 14 in relation to the tubular extension 38 and to secure the body 30 of the cervical cup 18 in relation to the inflatable balloon 16. It is envisioned that other locking devices may be provided to secure the cervical cup 18 in relation to the inflatable balloon 16.

The rim 34 of the body 30 of the cervical cup 18 supports a plurality of annularly spaced lights 36. In embodiments, the lights 34 may include light emitting diodes (LEDs) or any other known type of light suitable for internal use. The lights 36 face in the distal direction and are spaced along the rim 34 over the length of the rim 34. Although not shown, the lights 36 may be connected to a power source in the handle assembly 12 by one or more conductors, e.g., wires. For example, wires can be provided within the body 30 of the cervical cup 18 and extend through or within the tubular extension 38, or within the shaft 14, to the handle assembly 12. The handle assembly 12 may support a battery pack or include a socket adapted to connect to source of electrical energy. For example, the hand grip 12a may define a receptacle 25 configured to receive a battery 25a. A removable cap 27 may be supported on the hand grip 12a to enclose the receptacle 25.

Referring to FIG. 3, in use, the distal portion of the uterine manipulator 10 is inserted into the vaginal opening 50 by pushing shaft 14 distally. In this respect, the shaft 14 should be formed of a flexible material that has sufficient rigidity to allow the shaft to be pushed through the vaginal canal 52 without collapsing. In order to provide atraumatic translation of the cervical cup 18 through the vaginal canal 52, the inflatable balloon 16 is transitioned to the inflated state prior to insertion of the distal portion of the uterine manipulator 10 through the vaginal opening 50. In embodiments, the balloon 16 in its inflated state has an outer diameter slightly greater than the cervical cup 18 to shield the cervical cup 18 from substantial contact with walls defining the vaginal canal 52 during insertion of the distal portion of the uterine manipulator 10 into the vaginal canal 52. As the distal portion of the uterine manipulator 10 is inserted into the vaginal canal 52, visualization of the vaginal canal 52 is provided via the endoscope 20 through the clear tip 22 of the shaft 14.

Referring to FIG. 4, the distal portion of the uterine manipulator 10 is advanced within the vaginal canal 52 until the balloon approaches the cervix “C”. As the balloon 16 approaches the cervix “C”, as visualized through the endoscope 20 and tip 22 of the shaft 14, the balloon 16 is transitioned to its deflated state to facilitate entry of the distal portion of the uterine manipulator 10 into the uterus “U”. After the balloon 16 is deflated, the shaft 14 advanced distally to position the tip 22 within the uterus “U” and to position the rim 34 of the cervical cup 18 in abutting relation with the cervix “C”. When the rim 34 abuts the cervix “C”, the tubular extension 38 of the cervical cup 18 is disengaged from the shaft 14 by disengaging the locking device 21 from contact with the shaft 14. Upon disengaging the locking device 21 from engagement with the shaft 14, the shaft 14 is advanced independently of the cervical cup 18 to position the inflatable balloon 16 within the uterus “U”. The body 30 of the cervical cup 18 is also positioned to abut the cervix “C”. When the balloon 16 is properly positioned within the uterus “U”, the tubular extension 38 of the cervical cup 18 can be resecured to the shaft 14 using the locking device 21.

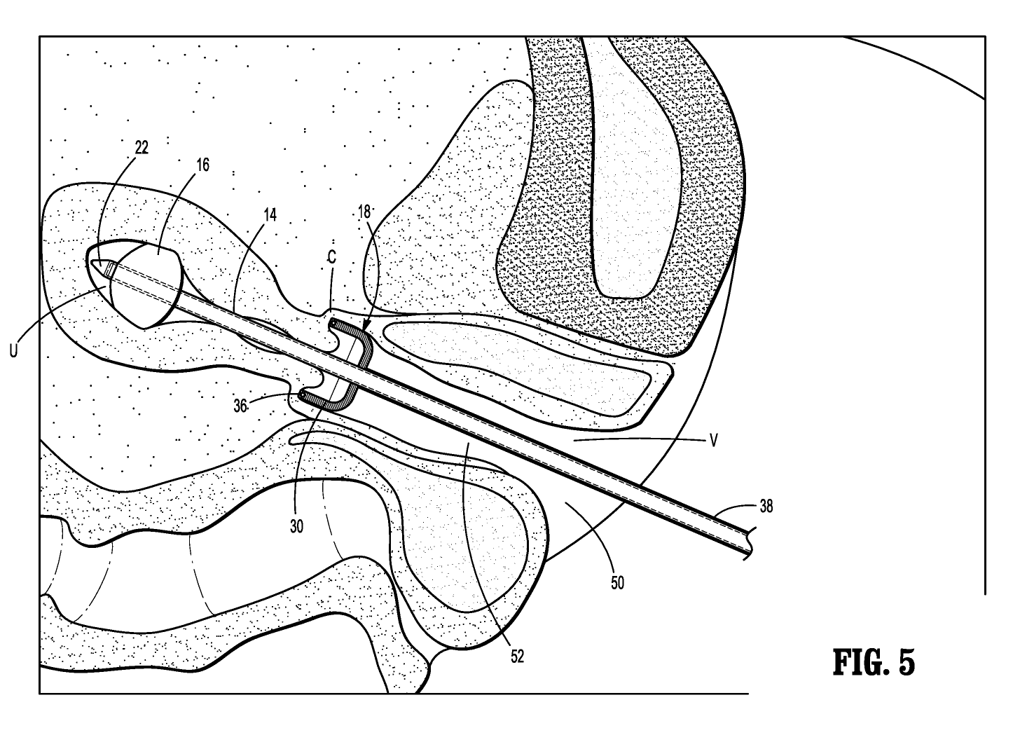

Referring to FIG. 5, when the balloon 16 is properly positioned within the uterus “U”, the balloon 16 is transitioned to the inflated state to secure the distal portion of the uterine manipulator 10 to the uterus “U”. After the balloon 16 is inflated, the uterine manipulator 10 can be manipulated to properly position the cervical cup 18 in relation to the cervix “C”.

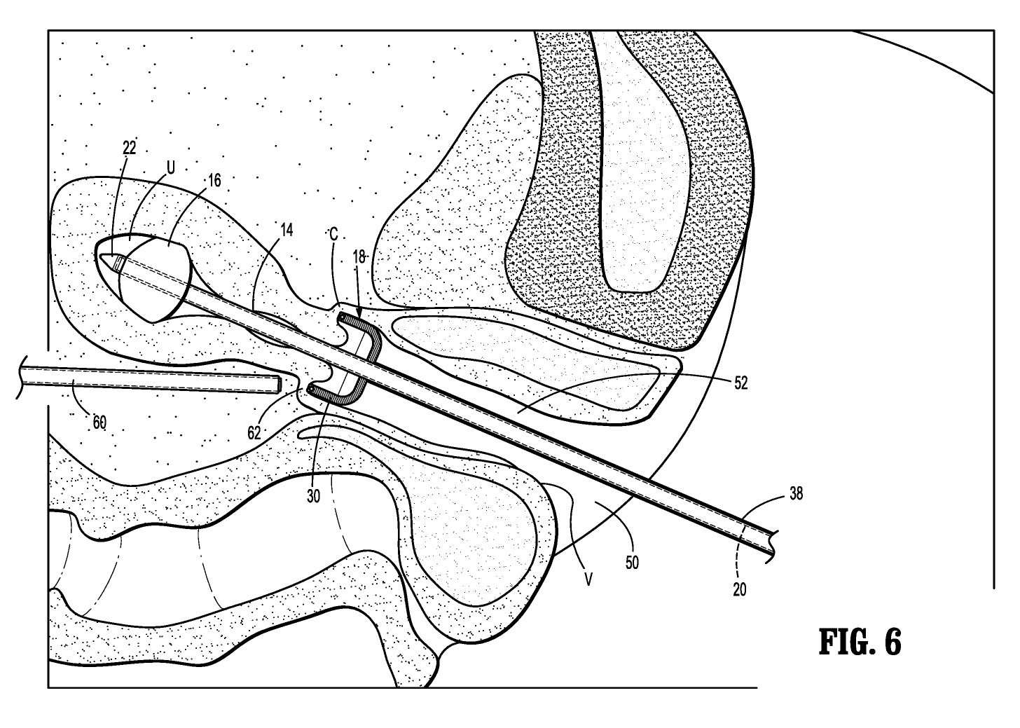

Referring also to FIG. 6, an endoscope 60 is inserted from the abdomen to a position to visualize the placement of the rim 34 of the cervical cup 18 at the posterior vaginal fornix 62. As discussed above, the rim 34 of the cervical cup 18 includes a plurality of lights 36 positioned about the rim 34. The lights 36 can be visualized through the cervix “C” by the abdominal endoscope 60 adjacent the vaginal fornix 62 to confirm proper placement of the cervical cup 18 and to identify the proper cutting location to the clinician. If the cervical cup 18 is not suitably positioned, the uterine manipulator 10 can be repositioned to reposition the rim 34 of the cervical cup 18 in relation to the cervix “C”.

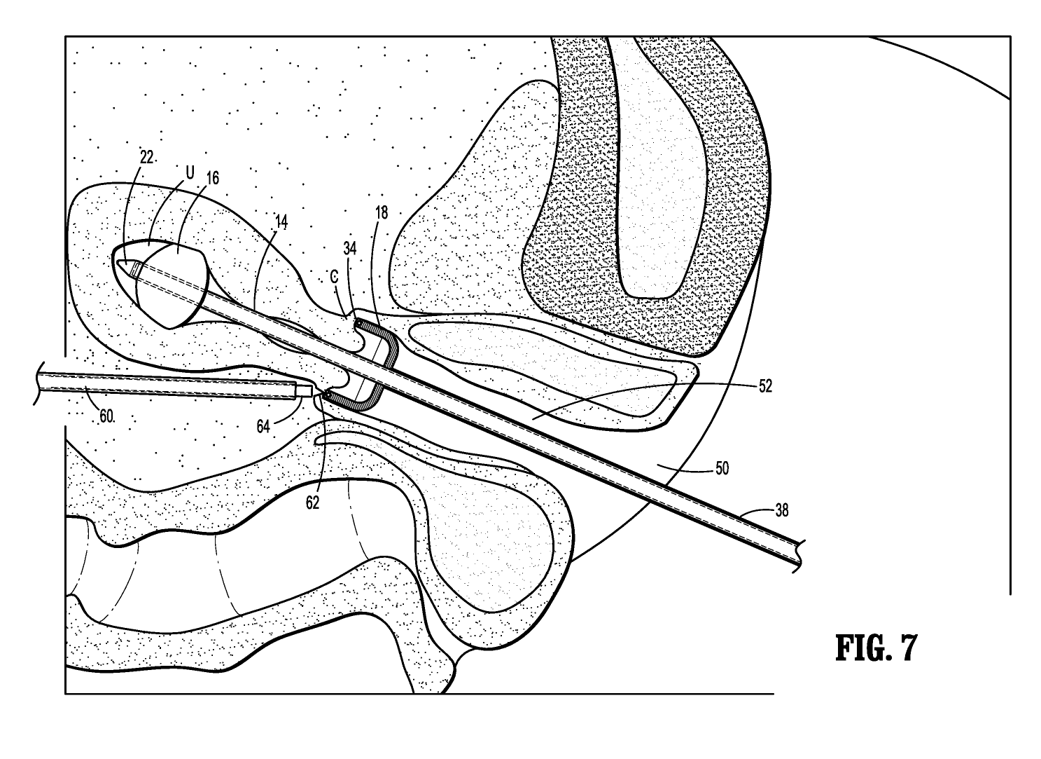

Referring to FIG. 7, after the body 18 of the cervical cup 18 is properly positioned in relation to the cervix “C”, a cutting instrument 64 is inserted through the abdominal endoscope 60 and a clinician can cut tissue directly over the lights 34 to accurately conduct a colpotomy and separate the uterus “U” from the cervix “C”. In embodiments, the surgical instrument 64 may be an electrically powered surgical device such as an ultrasonic cutting instrument or a RF cutting instrument. Each of these types of instruments effects simultaneous cutting and coagulation.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described embodiments. Accordingly, the disclosure is not to be limited by what has been particularly shown and described, except as indicated by the appended claims.

What is claimed is:

1. A uterine manipulator comprising:
an elongated, hollow shaft having a proximal end and a distal end, the hollow shaft defining a channel and having a non-opaque distal tip;
a handle assembly positioned on a proximal portion of the shaft;
an inflatable balloon supported on a distal portion of the shaft, the inflatable balloon being transitional from a deflated state to an inflated state; and
a cervical cup movably positioned along the shaft;
wherein the channel of the shaft is dimensioned to receive an endoscope to facilitate visualization of a body lumen through the non-opaque distal tip of the shaft.

2. The uterine manipulator of claim 1, wherein the cervical cup is movable along the shaft from a distal position in close approximation with the inflatable balloon to a proximal position spaced from the inflatable balloon.

3. The uterine manipulator of claim 2, wherein the outer diameter of the inflatable balloon is larger than the outer diameter the cervical cup.

4. The uterine manipulator of claim 2, wherein the cervical cup includes a body defining a cavity and a proximal extension positioned about the shaft and extending towards the handle assembly.

5. The uterine manipulator of claim 4, further including a first hand grip supported on the proximal portion of the shaft and a second hand grip supported on a proximal portion of the tubular extension, the first hand grip being movable in relation to the second hand grip to move the body of the cervical cup axially in relation to the inflatable balloon.

6. The uterine manipulator of claim 5, further including a locking device supported on the tubular extension of the cervical cup, the locking device being movable into contact with the shaft to axially secure the body of the cervical cup in relation to the inflatable balloon.

7. The uterine manipulator of claim 2, wherein the cervical cup includes a rim that is positioned to engage the inflatable balloon when the balloon is in the inflated state and the cervical cup is in the distal position.

8. The uterine manipulator of claim 7, wherein the rim supports a plurality of lights.

9. The uterine manipulator of claim 8, wherein the plurality of lights are positioned in an annular array about the rim.

10. The uterine manipulator of claim 1, wherein the inflatable balloon is lubricated.

11. A method of performing a surgical procedure comprising:
inserting a distal portion of a uterine manipulator into a patient's vaginal opening, the uterine manipulator including a hollow shaft defining a channel having a distal portion supporting a cervical cup, an inflatable balloon and a non-opaque distal tip, the cervical cup being proximal to the inflatable balloon;
visualizing a vaginal canal through the non-opaque distal tip with a first endoscope positioned within the hollow shaft;
advancing the inflatable balloon past the patient's cervix into the uterus; and
inflating the balloon within the uterus to secure the uterus to the distal portion of the uterine manipulator.

12. The method of claim 11, further including positioning the inflatable balloon in close approximation with the cervical cup prior to inserting the distal portion of the uterine manipulator into the patient's vaginal opening.

13. The method of claim 12, further including positioning the cervical cup in abutting relation to a cervix of the patient.

14. The method of claim 13, further including illuminating lights positioned on a distal rim of the cervical cup.

15. The method of claim 14, further including inserting a second endoscope through the patient's abdomen to a location to visualize the lights of the rim of the cervical cup at the posterior vaginal fornix.

16. The method of claim 15, further including inserting a cutting device through the second endoscope and cutting tissue directly over the lights to separate the patient's uterus from the cervix.

17. A uterine manipulator comprising:
an elongated shaft having a proximal end and a distal end;
a handle assembly positioned on a proximal portion of the shaft; and
a cervical cup movably positioned along the shaft from a distal position to a proximal position, the cervical cup including a body having a rim supporting a plurality of lights.

18. The uterine manipulator of claim 17, wherein the plurality of lights are positioned in an annular array about the rim.

19. The uterine manipulator of claim 18, further including an inflatable balloon supported on the shaft distally of the cervical cup, the inflatable balloon being transitional from a deflated state to an inflated state.

20. The uterine manipulator of claim 19, wherein the elongated shaft defines a channel and has a non-opaque distal tip, the channel of the elongated shaft being dimensioned to receive an endoscope to facilitate visualization of a body lumen through the non-opaque distal tip of the elongated shaft.

21. The uterine manipulator of claim 20, wherein the cervical cup defines a cavity and a tubular extension positioned about the shaft and extending towards the handle assembly, the rim of the cervical cup being positioned to engage the inflatable balloon when the balloon is in the inflated state and the cervical cup is in the distal position.

22. The uterine manipulator of claim 21, further including a first hand grip supported on the proximal portion of the elongated shaft and a second hand grip supported on a proximal portion of the tubular extension, the first hand grip being movable in relation to the second hand grip to move the body of the cervical cup axially in relation to the inflatable balloon.
